(19) United States
(12) Patent Application Publication
(10) Pub. No.: US 2012/0214891 A1
(43) Pub. Date: Aug. 23, 2012

(54) POLYESTER POLYOLS BASED ON AROMATIC DICARBOXYLIC ACIDS

(75) Inventors: Lionel GEHRINGER, Schaffhouse-pres-Seltz (FR); Gunnar KAMPF, Stemwede-Haldem (DE); Sirus ZARBAKHSH, Hong Kong (CN); Joachim-Thierry ANDERS, Dresden (DE)

(73) Assignee: BASF SE, Ludwigshafen (DE)

(21) Appl. No.: 13/400,835

(22) Filed: Feb. 21, 2012

Related U.S. Application Data
(60) Provisional application No. 61/445,573, filed on Feb. 23, 2011.

(51) Int. Cl. C08J 9/00; C07C 57/03; C08G 18/06

(52) U.S. Cl. 521/172; 528/83; 554/223; 554/227

(57) ABSTRACT

The present invention relates to polyester polyols based on aromatic dicarboxylic acids or derivatives thereof and to the use of the polyester polyols for producing polyurethanes.

The present invention relates to polyester polyols based on aromatic dicarboxylic acids and their use for producing rigid polyurethane foams.

The production of rigid polyurethane foams by reacting organic or modified organic diisocyanates or polyisocyanates with relatively high molecular weight compounds having at least two reactive hydrogen atoms, in particular with polyether polyols from alkylene oxide polymerization or polyester polyols from the polycondensation of alcohols with dicarboxylic acids, in the presence of polyurethane catalysts, chain extenders and/or crosslinkers, blowing agents and further auxiliaries and additives is known and is described in numerous patent and literature publications.

Mention may be made by way of example of the Kunststoffhandbuch, Volume VII, Polyurethane, Carl-Hanser-Verlag, Munich, 1st Edition 1966, edited by Dr. R. Vieweg and Dr. A. Höchtlen, and 2nd Edition 1983 and 3rd Edition 1993, edited by Dr. G. Oertel. Appropriate selection of the formative components and their ratios enables polyurethane foams having very good mechanical properties to be produced.

In the context of the present disclosure, the terms “polyester polyol”, “polyesterol”, “polyester alcohol” and the abbreviation “PESOL” are used synonymously. The abbreviation “PEOL” means polyetherol, which is equivalent to polyether alcohol. “Polyol” refers to a compound having at least two free OH groups.

When polyester polyols are used, it is usual to employ polycondensates of aromatic and/or aliphatic dicarboxylic acids and alkanediols and/or alkanetriols or ether diols. However, it is also possible to process polyester scrap, in particular polyethylene terephthalate (PET) or polybutylene terephthalate (PBT) scrap. A whole series of processes are known and have been described for this purpose. Some processes are based on the conversion of the polyester into a diester of terephthalic acid, e.g. dimethyl terephthalate. DE-A 1003714 and U.S. Pat. No. 5,051,528 describe such transesterifications using methanol and transesterification catalysts.

U.S. Pat. No. 3,138,562, which is concerned with “Cellular Polyurethane Plastics”, discloses a process for preparing polyester polyols in which trifunctional alcohols (TMP) are used. It is not said how the starting materials, for example polyether alcohols (propoxylated glycerol), are prepared.

It is also known that esters based on terephthalic acid are superior in terms of the burning behavior to esters based on phthalic acid. This is indicated, for example, in WO 2010/043624.

Polyether polyols can also be used for producing polyurethanes and also as component for the formation of polyesterols.

In general, these polyether polyols are prepared by catalyzed alkoxylation of an OH— functional starter.

Alkoxylation catalysts used are standard basic compounds, in particular KOH. Double metal cyanide (DMC) catalysts or carbenes are also used in some cases.

However, in the case of the KOH catalysts frequently used for the alkoxylation of OH— functional compounds, the reaction has to be followed by a work-up step in order to separate the catalyst from the product. This work-up is normally carried out by neutralization and subsequent filtration. However, small amounts of the product usually remain in the catalyst to be separated off and this decreases the product yield.

It would be desirable for the work-up of the polyetherols to be able to be dispensed with (because of, inter alia, polyol losses and the required investment in this plant part). However, in the case of the KOH catalysis routinely employed the basic catalyst would remain in the polyetherol and can hinder the subsequent acid-catalyzed esterification for producing polyesterols.

The additional time-consuming process step of work-up can be avoided when an amine catalyst is used in place of KOH. These amine catalysts can normally remain in the product.

However, if the polyetherol product is to be reacted further by esterification, e.g. with a dicarboxylic acid, to give a polyester polyol, the amine catalyst remaining in the polyetherol can likewise cause problems since it hinders the esterification which is usually catalyzed by a Lewis acid.

Furthermore, it is known that amine compounds generally reduce the hydrolysis stability of the polyesterol.

It is therefore an object of the present invention to provide a polyester polyol which is built up from, inter alia, a polyetherol and can be prepared with very little outlay (i.e. with, inter alia, very few work-up and purification steps) and in addition can be used without problems in polyurethane (PU) production and gives good results in the PU.

The abovementioned object was surprisingly able to be achieved by use of a polyether polyol component which can be prepared by reaction of a polyol, in particular a polyether alcohol, having a functionality of greater than or equal to 2 with at least one alkylene oxide in the presence of an amine catalyst in the polyester polyol.

The present invention accordingly provides a polyester polyol comprising the esterification product of
a. from 10 to 70 mol % of a dicarboxylic acid composition comprising
   a1) an amount of from 50 to 100 mol %, based on the dicarboxylic acid composition a), of an aromatic dicarboxylic acid or a mixture of aromatic dicarboxylic acids,
   a2) an amount of from 0 to 50 mol %, based on the dicarboxylic acid composition a), of one or more aliphatic dicarboxylic acids,
b. from 2 to 30 mol % of one or more fatty acids and/or fatty acid derivatives,
c. from 10 to 70 mol % of one or more aliphatic or cycloaliphatic diols having from 2 to 18 carbon atoms or alkoxylates thereof,
d. from 2 to 50 mol % of a polyether alcohol having a functionality of greater than or equal to 2 and prepared by alkoxylation of a polyol g) having a functionality of greater than or equal to 2 preferably greater than 2, in the presence of an amine as catalyst,
e. from 0 to 45 mol % of a polyether alcohol having a functionality of greater than or equal to 2 and prepared by alkoxylation of a polyol h) having a functionality of greater than or equal to 2 in the presence of a catalyst other than an amine,
f. from 0 to 50 mol % of a polyol having a functionality of greater than or equal to 2 selected from the group consisting of glycerol, trimethylolpropane, pentaerythritol,
   where the mol % of components a) to f) add up to 100% and at least 200 mmol, preferably at least 500 mmol and particularly preferably at least 800 mmol, of polyols d) having an OH functionality of greater than or equal to 2, preferably greater than or equal to 2.2 are reacted per kg of polyester polyol.

The present invention further provides a process for preparing a polyester polyol, wherein
a polyether alcohol (d) having a functionality of greater than or equal to 2 is prepared by alkoxylation of a polyol (g) having a functionality of greater than or equal to 2 in the presence of an amine as catalyst,
and a polyether alcohol (e) having a functionality of greater than or equal to 2 is optionally prepared by alkoxylation of a polyol (h) having a functionality of greater than or equal to 2 in the presence of a catalyst other than an amine and
from 2 to 50 mol % of the polyether alcohol (d) and
from 0 to 45 mol % of the polyether alcohol (e) and
from 0 to 50 mol % of a polyol (f) having a functionality of greater than or equal to 2 and selected from the group consisting of glycerol, trimethylolpropane, pentaerythritol, are reacted with
from 10 to 70 mol % of a dicarboxylic acid composition comprising (a1) from 50 to 100 mol %, based on the dicarboxylic acid composition (a), of an aromatic dicarboxylic acid or a mixture of aromatic dicarboxylic acids, (a2) from 0 to 50 mol %, based on the dicarboxylic acid composition (a), of one or more aliphatic dicarboxylic acids and
from 2 to 30 mol % of one or more fatty acids and/or fatty acid derivatives and
from 10 to 70 mol % of one or more aliphatic or cycloaliphatic diols having from 2 to 18 carbon atoms or alkoxylates thereof,
   where the mol % of components (a) to (f) add up to 100% respectively and at least 200 mmol of polyols (d) having an OH functionality of greater than or equal to 2 are reacted per kg of polyester polyol.

In one embodiment of the present invention, the component a1) comprises at least one material from the group consisting of terephthalic acid, dimethyl terephthalate (DMT), polyethylene terephthalate (PET), phthalic acid, phthalic anhydride (PAn) and isophthalic acid.

In one embodiment of the present invention, the component a1) comprises at least one material from the group consisting of terephthalic acid, dimethyl terephthalate (DMT), polyethylene terephthalate (PET) and phthalic anhydride (PAn), preferably terephthalic acid, dimethyl terephthalate (DMT) and polyethylene terephthalate (PET), particularly preferably terephthalic acid.

In one embodiment of the present invention, the aliphatic dicarboxylic acid a2) is selected from the group consisting of aliphatic dicarboxylic acids having from 4 to 6 carbon atoms.

In one embodiment of the present invention, the component a2) is comprised in an amount of from 0 to 30 mol %, preferably from 0 to 10 mol %, particularly preferably 0 mol %, in the dicarboxylic acid composition a).

In one embodiment of the present invention, the component b) goes into the esterification product to an extent of from 3 to 20 mol %, particularly preferably from 5 to 18 mol %.

In one embodiment of the present invention, the component c) goes into the esterification product to an extent of from 20 to 60 mol %, preferably from 25 to 55 mol %, particularly preferably from 30 to 40 mol %.

In one embodiment of the present invention the component d) goes into the esterification product to an extent of from 2 to 40 mol %, preferably from 2 to 35 mol %, particularly preferably from 20 to 25 mol %.

In a preferred embodiment of the present invention, the amine catalyst in d) is selected from the group consisting of DMEOA (dimethylethanolamine), imidazole and imidazole derivatives and mixtures thereof, particularly preferably imidazole.

In one embodiment of the present invention, the catalyst other than an amine in e) is selected from the group consisting of KOH, double metal cyanide (DMC) catalysts, carbenes.

In one embodiment of the present invention, the aliphatic or cycloaliphatic diol c) is selected from the group consisting of ethylene glycol, diethylene glycol and propylene glycol, 1,3-propanediol, 1,4-butanediol, 1,5-pentanediol, 1,6-hexanediol, 2-methyl-1,3-propanediol, 3-methyl-1,5-pentanediol and alkoxylates thereof.

In a preferred embodiment of the present invention, the aliphatic diol c) is diethylene glycol.

In one embodiment of the present invention, the fatty acid or the fatty acid derivative b) is a fatty acid or fatty acid derivative based on renewable resources and is selected from the group consisting of castor oil, polyhydroxy fatty acids, ricinoleic acid, hydroxyl-modified oils, grapeseed oil, black cumin oil, pumpkin kernel oil, borage seed oil, soybean oil, wheat germ oil, rapeseed oil, sunflower oil, peanut oil, apricot kernel oil, pistachio oil, almond oil, olive oil, macadamia nut oil, avocado oil, sea buckthorn oil, sesame oil, hemp oil, hazelnut oil, primula oil, wild rose oil, safflower oil, walnut oil, hydroxyl-modified fatty acids and fatty acid esters based on myristoleic acid, palmitoleic acid, oleic acid, vaccenic acid, petroselic acid, gadoleic acid, erucic acid, nervonic acid, linoleic acid, α- and γ-linolenic acid, stearidonic acid, arachidonic acid, timnodonic acid, clupanodonic acid and cervonic acid.

In a preferred embodiment of the present invention, the fatty acid or the fatty acid derivative b) is oleic acid and/or soybean oil and/or rapeseed oil, particularly preferably oleic acid.

In one embodiment of the present invention, the polyether alcohol d) having a functionality of greater than or equal to 2 is selected from the group consisting of reaction products of glycerol, trimethylolpropane (TMP), pentaerythritol and polyethylene glycol (PEG) and mixtures thereof with an alkylene oxide.

In one embodiment of the present invention, the polyether alcohol d) having a functionality of greater than or equal to 2 is prepared by reacting a polyol e) having a functionality of greater than or equal to 2 with ethylene oxide and/or propylene oxide, preferably with ethylene oxide.

In one embodiment of the present invention, the polyether alcohol d) having a functionality of greater than or equal to 2 comprises the reaction product of glycerol with ethylene oxide and/or propylene oxide, preferably with ethylene oxide.

In one embodiment of the present invention, the polyether alcohol d) having a functionality of greater than or equal to 2 comprises the reaction product of trimethylolpropane with ethylene oxide and/or propylene oxide, preferably with ethylene oxide.

In one embodiment of the present invention, the polyether alcohol d) having a functionality of greater than or equal to 2 has an OH number in the range from 1250 to 100 mg KOH/g, preferably from 950 to 150 mg KOH/g, particularly preferably from 800 to 240 mg KOH/g.

In a preferred embodiment of the present invention, the polyether alcohol d) having a functionality of greater than or equal to 2 comprises the reaction product of trimethylolpropane or glycerol with ethylene oxide, where the OH number of the polyether alcohol d) is in the range from 500 to 650 mg KOH/g.

In a particularly preferred embodiment of the present invention, the polyether alcohol d) having a functionality of greater than or equal to 2 comprises the reaction product of trimethylolpropane or glycerol with ethylene oxide, where the OH number of the polyether alcohol d) is in the range from 500 to 625 mg KOH/g, and the aliphatic or cycloaliphatic diol c) is diethylene glycol and the fatty acid or fatty acid derivative is oleic acid.

In one embodiment of the present invention, a polyether alcohol d) which has a functionality of greater than 2 and has been prepared by alkoxylation of a polyol ge) having a functionality of greater than or equal to 3 is used.

To prepare the polyester polyols of the invention, the organic, e.g. aliphatic and preferably aromatic, polycarboxylic acids and/or derivatives and polyhydric alcohols are polycondensed in the absence of catalysts or preferably in the presence of esterification catalysts, advantageously in an atmosphere of inert gas, e.g. nitrogen, carbon monoxide, helium, argon, etc., in the melt at temperatures of from 150 to 280° C., preferably from 180 to 260° C., optionally under reduced pressure, to the desired acid number which is advantageously less than 10, preferably less than 2. In a preferred embodiment, the esterification mixture is polycondensed at the abovementioned temperatures to an acid number of from 80 to 20, preferably from 40 to 20, under atmospheric pressure and subsequently under a pressure of less than 500 mbar, preferably from 40 to 400 mbar. Possible esterification catalysts are, for example, iron, cadmium, cobalt, lead, zinc, antimony, magnesium, titanium and tin catalysts in the form of metals, metal oxides or metal salts. However, the polycondensation can also be carried out in the liquid phase in the presence of diluents and/or entrainers such as, benzene, toluene, xylene or chlorobenzene in order to distill off the water of condensation as an azeotrope.

To prepare the polyester polyols, the organic polycarboxylic acids and/or derivatives and polyhydric alcohols are advantageously polycondensed in a molar ratio of 1:1-2.1, preferably 1:1.05-2.0.

The polyester polyols obtained preferably have a functionality of from 1.8 to 4, in particular from 2 to 3, and a molecular weight of from 300 to 3000, preferably from 400 to 1000 and in particular from 450 to 800.

In a preferred embodiment of the present invention, the polyester polyol of the invention has an average functionality of greater than or equal to 2, preferably greater than 2.2.

Furthermore, the invention also provides a process for producing rigid PU foams.

In particular, the invention provides a process for producing rigid polyurethane foams by reacting
A. at least one organic and/or modified organic diisocyanate and/or polyisocyanate with
B. at least one specific polyester polyol according to the invention,
C. optionally at least one further polyester polyol,
D. optionally at least one polyetherol and/or at least one further compound having at least two groups which are reactive toward isocyanates,
E. optionally at least one chain extender and/or crosslinker,
F. at least one blowing agent,
G. at least one catalyst and also
H. optionally at least one further auxiliary and/or at least one additive and
I. optionally at least one flame retardant.

The present invention further provides rigid polyurethane foams and rigid polyisocyanurate foams which can be obtained by the process of the invention, and also the use of the polyester polyols of the invention for producing rigid polyurethane foams or rigid polyisocyanurate foams.

To produce the rigid polyurethane foams by the process of the invention, use is made of, in addition to the above-described specific polyester polyols, the formative components which are known per se, about which the following details may be provided.

Possible organic and/or modified organic polyisocyanates A) are the aliphatic, cycloaliphatic, araliphatic and preferably aromatic polyfunctional isocyanates known per se.

Specific examples are: alkylene diisocyanates having from 4 to 12 carbon atoms in the alkylene radical, e.g. dodecane 1,12-diisocyanate, 2-ethyltetramethylene 1,4-diisocyanate, 2-methylpentamethylene 1,5-diisocyanate, tetramethylene 1,4-diisocyanate, and preferably hexamethylene 1,6-diisocyanate; cycloaliphatic diisocyanates such as cyclohexane 1,3- and 1,4-diisocyanate and also any mixtures of these isomers, 1-isocyanato-3,3,5-trimethyl-5-isocyanatomethylcyclohexane (IPDI), hexahydrotolylene 2,4- and 2,6-diisocyanate and also the corresponding isomer mixtures, dicyclohexylmethane 4,4′-, 2,2′- and 2,4′-diisocyanate and also the corresponding isomer mixtures and preferably aromatic diisocyanates and polyisocyanates such as tolylene 2,4- and 2,6-diisocyanate and the corresponding isomer mixtures, diphenylmethane 4,4′-, 2,4′- and 2,2′-diisocyanate and the corresponding isomer mixtures, mixtures of diphenylmethane 4,4′- and 2,2′-diisocyanates, polyphenylpolymethylene polyisocyanates, mixtures of diphenylmethane 2,4′-, 2,4′- and 2,2′-diisocyanates and polyphenylpolymethylene polyisocyanates (crude MDI) and mixtures of crude MDI and tolylene diisocyanates. The organic diisocyanates and polyisocyanates can be used individually or in the form of their mixtures.

Preferred diisocyanates and polyisocyanates are tolylene diisocyanate (TDI), diphenylmethane diisocyanate (MDI) and in particular mixtures of diphenylmethane diisocyanate and polyphenylenepolymethylene polyisocyanates (polymeric MDI or PMDI).

Use is frequently also made of modified polyfunctional isocyanates, i.e. products which are obtained by chemical reaction of organic diisocyanates and/or polyisocyanates. Examples which may be mentioned are diisocyanates and/or polyisocyanates comprising ester, urea, biuret, allophanate, carbodiimide, isocyanurate, uretdione, carbamate and/or urethane groups.

Very particular preference is given to using polymeric MDI for producing rigid polyurethane foams.

Suitable further polyester polyols C) can be prepared, for example, from organic dicarboxylic acids having from 2 to 12 carbon atoms, preferably aliphatic dicarboxylic acids having 4-6 carbon atoms, and polyhydric alcohols, preferably diols, having from 2 to 12 carbon atoms, preferably from 2 to 6 carbon atoms. Possible dicarboxylic acids are, for example: succinic acid, glutaric acid, adipic acid, suberic acid, azelaic acid, sebacic acid, decanedicarboxylic acid, maleic acid, fumaric acid, phthalic acid, isophthalic acid and terephthalic acid. The dicarboxylic acids can be used either individually or in admixture with one another. It is also possible to use the corresponding dicarboxylic acid derivatives, e.g. dicarboxylic esters of alcohols having from 1 to 4 carbon atoms or dicarboxylic anhydrides, in place of the free dicarboxylic acids. Preference is given to using dicarboxylic acid mixtures of succinic, glutaric and adipic acid in weight ratios of, for example, 20-35:35-50:20-32 and in particular adipic acid. Examples of dihydric and polyhydric alcohols, in particular diols, are: ethanediol, diethylene glycol, 1,2- or 1,3-propanediol, dipropylene glycol, 1,4-butanediol, 1,5-pentanediol, 1,6-hexanediol, 1,10-decanediol, glycerol, trimethylolpropane and pentaerythritol. Preference is given to using ethanediol, diethylene glycol, 1,4-butanediol, 1,5-pentanediol, 1,6-hexanediol or mixtures of at least two of the diols mentioned, in particular mixtures of 1,4-butanediol, 1,5-pentanediol and 1,6-hexanediol. It is also possible to use polyester polyols derived from lactones, e.g. ε-caprolactone, or hydroxycarboxylic acids, e.g. ω-hydroxycaproic acid.

It is also possible to make concomitant use of polyether polyols D) which are prepared by known methods, for example from one or more alkylene oxides having from 2 to 4 carbon atoms in the alkylene radical by anionic polymerization using alkali metal hydroxides, e.g. sodium or potassium hydroxide, or alkali metal alkoxides, e.g. sodium methoxide, sodium or potassium ethoxide or potassium isopropoxide, as catalysts with addition of at least one starter molecule comprising from 2 to 8, preferably from 2 to 6, reactive hydrogen atoms, or by cationic polymerization using Lewis acids, e.g. antimony pentachloride, boron fluoride etherate, etc., or bleaching earth, as catalysts.

Suitable alkylene oxides are, for example, tetrahydrofuran, 1,3-propylene oxide, 1,2- or 2,3-butylene oxide, styrene oxide and preferably ethylene oxide and 1,2-propylene oxide. The alkylene oxides can be used individually, alternately in succession or as mixtures. Preferred alkylene oxides are propylene oxide and ethylene oxide, with particular preference being given to ethylene oxide.

Possible starter molecules are, for example: water, organic dicarboxylic acids, such as succinic acid, adipic acid, phthalic acid and terephthalic acid, aliphatic and aromatic, unsubstituted or N-monoalkyl-, N,N-dialkyl- and N,N′-dialkyl-substituted diamines having from 1 to 4 carbon atoms in the alkyl radical, e.g. unsubstituted or monoalkyl- and dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexa-methylenediamine, phenylenediamines, 2,3-, 2,4- and 2,6-toluenediamine and 4,4′-, 2,4′- and 2,2′-diaminodiphenylmethane.

Further possible starter molecules are: alkanolamines such as ethanolamine, N-methylethanolamine and N-ethylethanolamine, dialkanolamines, such as diethanolamine, N-methyldiethanolamine and N-ethyldiethanolamine and trialkanolamines, such as triethanolamine, and ammonia. Preference is given to using dihydric or polyhydric alcohols such as ethanediol, 1,2- and 1,3-propanediol, diethylene glycol, dipropylene glycol, 1,4-butanediol, 1,6-hexanediol, glycerol, trimethylolpropane, pentaerythritol, sorbitol and sucrose.

The polyether polyols, preferably polyoxyethylene polyols, have a functionality of preferably from 2 to 6 and in particular from 2 to 5 and molecular weights of from 300 to 3000, preferably from 300 to 2000 and in particular from 400 to 1000.

Further suitable polyether polyols are polymer-modified polyether polyols, preferably graft polyether polyols, in particular those based on styrene and/or acrylonitrile which are prepared by in-situ polymerization of acrylonitrile, styrene or preferably mixtures of styrene and acrylonitrile, e.g. in a weight ratio of from 90:10 to 10:90, preferably from 70:30 to 30:70, advantageously in the abovementioned polyether polyols using methods analogous to those described in the German patent texts 11 11 394, 12 22 669 (U.S. Pat. Nos. 3,304,273, 3,383,351, 3,523,093), 11 52 536 (GB 10 40 452) and 11 52 537 (GB 987,618), and also polyether polyol dispersions which comprise, for example, polyureas, polyhydrazides, polyurethanes comprising bound tert-amino groups and/or melamine as disperse phase, usually in an amount of from 1 to 50% by weight, preferably from 2 to 25% by weight, and are described, for example, in EP-B 011 752 (U.S. Pat. No. 4,304,708), U.S. Pat. No. 4,374,209 and DE-A,32 31 497.

Like the polyester polyols, the polyether polyols can be used individually or in the form of mixtures. They can also be mixed with the graft polyether polyols or polyester polyols and with the hydroxyl-comprising polyesteram ides, polyacetals, polycarbonates and/or polyether polyamines.

Possible hydroxyl-comprising polyacetals are, for example, the compounds which can be prepared from glycols such as diethylene glycol, triethylene glycol, 4,4′-dihydroxy-ethoxydiphenyldimethylmethane, hexanediol and formaldehyde. Suitable polyacetals can also be prepared by polymerization of cyclic acetals.

Possible hydroxyl-comprising polycarbonates are those of the type known per se which can be prepared, for example, by reacting diols such as 1,3-propanediol, 1,4-butanediol and/or 1,6-hexanediol, diethylene glycol, triethylene glycol or tetraethylene glycol with diaryl carbonates, e.g. diphenyl carbonate, alkylene carbonate or phosgene.

The polyesteramides include, for example, the predominantly linear condensates obtained from polybasic, saturated and/or unsaturated carboxylic acids or anhydrides thereof and polyhydric saturated and/or unsaturated amino alcohols or mixtures of polyhydric alcohols and amino alcohols and/or polyamines.

Suitable polyether polyamines can be prepared from the abovementioned polyether polyols by known methods. Mention may be made by way of example of the cyanoalkylation of polyoxyalkylene polyols and subsequent hydrogenation of the nitrile formed (US 3 267 050) or the partial or complete amination of polyoxyalkylene polyols with amines or ammonia in the presence of hydrogen and catalysts (DE 12 15 373).

The rigid polyurethane foams can be produced using chain extenders and/or crosslinkers (E). However, the addition of chain extenders, crosslinkers or, optionally, mixtures thereof can prove to be advantageous for modifying the mechanical properties, e.g. the hardness. As chain extenders and/or crosslinkers, use is made of diols and/or triols having molecular weights of less than 400, preferably from 60 to 300. Possibilities are, for example, aliphatic, cycloaliphatic and/or araliphatic diols having from 2 to 14, preferably from 4 to 10 carbon atoms, e.g. ethylene glycol, 1,3-propanediol, 1,10-decanediol, o-, m-, p-dihydroxycyclohexane, diethylene glycol, dipropylene glycol and preferably 1,4-butanediol, 1,6-hexanediol and bis(2-hydroxy-ethyl)hydroquinone, triols such as 1,2,4-, 1,3,5-trihydroxycyclohexane, glycerol and trimethylolpropane and low molecular weight hydroxyl-comprising polyalkylene oxides based on ethylene oxide and/or 1,2-propylene oxide and the abovementioned diols and/or triols as starter molecules.

Possible further compounds (D) having at least two groups which are reactive toward isocyanate, i.e. having at least two hydrogen atoms which are reactive toward isocyanate groups, are in particular those which have two or more reactive groups selected from among OH groups, SH groups, NH groups, NH₂ groups and CH-acid groups, e.g. β-diketo groups.

If chain extenders, crosslinkers or mixtures thereof are employed for producing the rigid polyurethane foams, they are advantageously used in an amount of from 0 to 20% by weight, preferably from 0.5 to 5% by weight, based on the weight of the component B).

Blowing agents F) which are used for producing the rigid polyurethane foams include preferably water, formic acid and mixtures thereof. These react with isocyanate groups to form carbon dioxide and in the case of formic acid carbon dioxide and carbon monoxide. In addition, physical blowing agents such as low-boiling hydrocarbons can be used. Suitable physical blowing agents are liquids which are inert towards the organic, modified or nonmodified polyisocyanates and have boiling points below 100° C., preferably below 50° C., at atmospheric pressure, so that they vaporize under the conditions of the exothermic polyaddition reaction. Examples of such liquids which can preferably be used are alkanes such as heptane, hexane, n-pentane and isopentane, preferably industrial mixtures of n-pentane and isopentane, n-butane and isobutane and propane, cycloalkanes such as cyclopentane and/or cyclohexane, ethers such as furan, dimethyl ether and diethyl ether, ketones such as acetone and methyl ethyl ketone, alkyl carboxylates such as methyl formate, dimethyl oxalate and ethyl acetate and halogenated hydrocarbons such as methylene chloride, dichloromono-fluoromethane, difluoromethane, trifluoromethane, difluoroethane, tetrafluoroethane, chlorodifluoroethanes, 1,1-dichloro-2,2,2-trifluoroethane, 2,2-dichloro-2-fluoroethane and heptafluoropropane. Mixtures of these low-boiling liquids with one another and/or with other substituted or unsubstituted hydrocarbons can also be used. Organic carboxylic acids such as formic acid, acetic acid, oxalic acid, ricinoleic acid and carboxyl-comprising compounds are also suitable.

Preference is given to using water, formic acid, chlorodifluoromethane, chlorodifluoroethanes, dichlorofluoroethanes, all pentane isomers and mixtures thereof, cyclohexane and mixtures of at least two of these blowing agents, e.g. mixtures of water and cyclohexane, mixtures of chlorodifluoromethane and 1-chloro-2,2-difluoroethane and optionally water.

The blowing agents are either completely or partly dissolved in the polyol component (i.e. B+C+E+F+G+H+I) or are introduced via a static mixer immediately before foaming of the polyol component. It is usual for water or formic acid to be fully or partially dissolved in the polyol component and the physical blowing agent (for example pentane) and optionally the remainder of the chemical blowing agent to be introduced “on-line”.

The amount of blowing agent or blowing agent mixture used is from 1 to 45% by weight, preferably from 1 to 30% by weight, particularly preferably from 1.5 to 20% by weight, in each case based on the sum of the components B) to G).

If water serves as blowing agent, it is preferably added to the formative component B) in an amount of from 0.2 to 5% by weight, based on the formative component B). The addition of water can be combined with the use of the other blowing agents described.

Catalysts G) used for producing the rigid polyurethane foams are, in particular, compounds which strongly accelerate the reaction of the compounds comprising reactive hydrogen atoms, in particular hydroxyl groups, of component B) and optionally C) with the organic, modified or nonmodified polyisocyanates A).

It is advantageous to use basic polyurethane catalysts, for example tertiary amines such as triethylamine, tributylamine, dimethylbenzylamine, dicyclohexylmethylamine, dimethylcyclohexylamine, bis(N,N-dimethylaminoethyl) ether, bis(dimethylamino-propyl)urea, N-methylmorpholine or N-ethylmorpholine, N-cyclohexylmorpholine, N,N,N′,N′-tetramethylethylenediamine, N, N,N′,N′-tetramethylbutanediamine, N,N,N′,N′-tetramethylhexane-1,6-diamine, pentamethyldiethylenetriamine, dimethyl-piperazine, N-dimethylaminoethylpiperidine, 1,2-dimethylimidazole, 1-azabicyclo-[2.2.0]octane, 1,4-diazabicyclo[2.2.2]octane (Dabco) and alkanolamine compounds, such as triethanolamine, triisopropanolamine, N-methyldiethanolamine and N-ethyl-diethanolamine, dimethylaminoethanol, 2-(N,N-dimethylaminoethoxy)ethanol, N,N′,N″-tris(dialkylaminoalkyl)hexahydrotriazines, e.g. N,N′,N″-tris(dimethylamino-propyl)-s-hexahydrotriazine, and triethylenediamine. However, metal salts such as iron(II) chloride, zinc chloride, lead octoate and preferably tin salts such as tin dioctoate, tin diethylhexoate and dibutyltin dilaurate and also, in particular, mixtures of tertiary amines and organic tin salts are also suitable.

Further possible catalysts are: amidines such as 2,3-dimethyl-3,4,5,6-tetra-hydropyrimidine, tetraalkylammonium hydroxides such as tetramethylammonium hydroxide, alkali metal hydroxides such as sodium hydroxide and alkali metal alkoxides such as sodium methoxide and potassium isopropoxide and also alkali metal salts of long-chain fatty acids having from 10 to 20 carbon atoms and optionally lateral OH groups. Preference is given to using from 0.001 to 5% by weight, in particular from 0.05 to 2% by weight, of catalyst or catalyst combination, based on the weight of the component B). It is also possible to allow the reactions to proceed without catalysis. In this case, the catalytic activity of amine-initiated polyols is exploited.

If, during foaming, a relatively large polyisocyanate excess is used, further suitable catalysts for the trimerization reaction of the excess NCO groups with one another are: catalysts which form isocyanurate groups, for example ammonium salts or alkali metal salts either alone or in combination with tertiary amines. Isocyanurate formation leads to flame-resistant PIR foams which are preferably used in industrial rigid foam, for example in building and construction as insulation boards or sandwich elements. In the following, the term polyurethanes will also include the foam class of PIR foams.

Further information regarding the abovementioned and further starting materials may be found in the technical literature, for example Kunststoffhandbuch, Volume VII, Polyurethane, Carl Hanser Verlag Munich, Vienna, 1st, 2nd and 3rd Editions 1966, 1983 and 1993.

Further auxiliaries and/or additives H) can optionally be added to the reaction mixture for producing the rigid polyurethane foams. Mention may be made of, for example, surface-active substances, foam stabilizers, cell regulators, fillers, dyes, pigments, flame retardants, hydrolysis inhibitors, fungistatic and bacteriostatic substances.

Possible surface-active substances are, for example, compounds which serve to aid homogenization of the starting materials and may also be suitable for regulating the cell structure of the polymers. Mention may be made of, for example, emulsifiers such as the sodium salts of castor oil sulfates or of fatty acids and also salts of fatty acids with amines, e.g. diethylamine oleate, diethanolamine stearate, diethanolamine ricinoleate, salts of sulfonic acids, e.g. alkali metal or ammonium salts of dodecylbenzenesulfonic or dinaphthylmethanedisulfonic acid and ricinoleic acid; foam stabilizers such as siloxane-oxyalkylene copolymers and other organopolysiloxanes, ethoxylated alkylphenols, ethoxylated fatty alcohols, paraffin oils, castor oil esters or ricinoleic esters, Turkey red oil and peanut oil, and cell regulators such as paraffins, fatty alcohols and dimethylpolysiloxanes. The above-described oligomeric acrylates having polyoxyalkylene and fluoroalkane radicals as side groups are also suitable for improving the emulsifying action, the cell structure and/or for stabilizing the foam. The surface-active substances are usually employed in amounts of from 0.01 to 10% by weight, based on 100% by weight of the component B).

For the purposes of the present invention, fillers, in particular reinforcing fillers, are the customary organic and inorganic fillers, reinforcing materials, weighting agents, agents for improving the abrasion behavior in paints, coating compositions, etc., which are known per se. Specific examples are: inorganic fillers such as siliceous minerals, for example sheet silicates such as antigorite, serpentine, hornblendes, amphiboles, chrisotile and talc, metal oxides such as kaolin, aluminum oxides, titanium oxides and iron oxides, metal salts, such as chalk, barite and inorganic pigments such as cadmium sulfide and zinc sulfide and also glass, etc. Preference is given to using kaolin (china clay), aluminum silicate and coprecipitates of barium sulfate and aluminum silicate and also natural and synthetic fibrous minerals such as wollastonite, metal fibers and in particular glass fibers of various length, which may be coated with a size. Possible organic fillers are, for example: carbon, melamine, rosin, cyclopentadienyl resins and graft polymers and also cellulose fibers, polyamide, polyacrylonitrile, polyurethane, polyester fibers based on aromatic and/or aliphatic dicarboxylic esters and in particular carbon fibers.

The inorganic and organic fillers can be used individually or as mixtures and are advantageously added to the reaction mixture in amounts of from 0.5 to 50% by weight, preferably from 1 to 40% by weight, based on the weight of the components A) to C), although the content of mats, nonwovens and woven fabrics of natural and synthetic fibers can reach values of up to 80% by weight.

As flame retardants I), it is generally possible to use the flame retardants known from the prior art. Suitable flame retardants are, for example, unincorporatable brominated substances, brominated esters, brominated ethers (Ixol) or brominated alcohols such as dibromoneopentyl alcohol, tribromoneopentyl alcohol and PHT-4-diol and also chlorinated phosphates such as tris(2-chloroethyl)phosphate, tris(2-chloropropyl)phosphate, tris(1,3-dichloropropyl)phosphate, tricresyl phosphate, tris(2,3-dibromo-propyl)phosphate, tetrakis(2-chloroethyl)ethylenediphosphate, dimethyl methanephosphonate, diethyl diethanolaminomethylphosphonate and also commercial halogen-comprising flame retardant polyols. As further liquid flame retardants, it is possible to use phosphates or phosphonates, e.g. diethyl ethanephosphonate (DEEP), triethylphosphate (TEP), dimethyl propylphosphonate (DMPP), diphenyl cresyl phosphate (DPK) and others.

Apart from the abovementioned flame retardants, it is possible to use inorganic or organic flame retardants such as red phosphorus, preparations comprising red phosphorus, aluminum oxide hydrate, antimony trioxide, arsenic oxide, ammonium polyphosphate and calcium sulfate, expandable graphite or cyanuric acid derivatives such as melamine, or mixtures of at least two flame retardants, e.g. ammonium polyphosphates and melamine and optionally maize starch or ammonium polyphosphate, melamine and expandable graphite and/or optionally aromatic polyesters for making the rigid polyurethane foams flame resistant.

In general, it has been found to be advantageous to use from 5 to 150% by weight, preferably from 10 to 100% by weight, of the flame retardants mentioned, based on the component B).

Further information regarding the abovementioned other customary auxiliaries and additives may be found in the technical literature, for example the monograph by J. H. Saunders and K. C. Frisch “High Polymers” Volume XVI, Polyurethanes, Parts 1 and 2, Interscience Publishers 1962 and 1964, or Kunststoff-Handbuch, Polyurethane, Volume VII, Hanser-Verlag, Munich, Vienna, 1st and 2nd Editions, 1966 and 1983.

To produce the rigid polyurethane foams of the invention, the organic and/or modified organic polyisocyanates A), the specific polyester polyols B) of the invention and optionally polyetherols and/or further compounds D) having at least two groups which are reactive toward isocyanates and optionally chain extenders and/or crosslinkers E) are reacted in such amounts that the equivalence ratio of NCO groups of the polyisocyanates A) to the sum of the reactive hydrogen atoms of the components B) and optionally C) and D) to G) is 1-6:1, preferably 1.1-5:1 and in particular 1.2-3.5:1.

The rigid polyurethane foams are advantageously produced by the one-shot process, for example by means of the high-pressure or low-pressure technique, in open or closed molds, for example metallic molds. Continuous application of the reaction mixture to suitable conveyor belts for producing panels is also customary.

The starting components are mixed at a temperature of from 15 to 90° C., preferably from 20 to 60° C. and in particular from 20 to 35° C., and introduced into the open mold or, optionally under elevated pressure, into the closed mold or, in the case of a continuous workstation, applied to a belt which accommodates the reaction mixture. Mixing can, as indicated above, be carried out mechanically by means of a stirrer or a stirring screw. The mold temperature is advantageously from 20 to 110° C., preferably from 30 to 70° C. and in particular from 40 to 60° C.

The rigid polyurethane foams produced by the process of the invention have a density of from 15 to 300 g/I, preferably from 20 to 100 g/I and in particular from 25 to 60 g/I.

### EXAMPLES

The present invention is illustrated by the following examples, with the examples only serving to illustrate certain aspects of the invention and in no way being intended as a limitation of the scope of the invention.

Various polyesterols were prepared:

General method for preparing the polyester polyol

The dicarboxylic acid, the fatty acid of the fat derivative, the aliphatic or cycloaliphatic diol or alkoxylates thereof and the higher-functional polyol were introduced into a 4 liter round-bottom flask equipped with a mechanical stirrer, a thermometer and a distillation column and also a nitrogen inlet tube. After addition of 300 ppm of titanium tetrabutylate as catalyst, the mixture is stirred and heated to 240° C., with the water liberated being distilled off continuously. The reaction is carried out at 400 mbar. This gives a polyesterol having an acid number of 1 mg KOH/g.

General method for the amine-catalyzed preparation of a polyetherol (polyetherol in (d) of claim 1)

A 960 l pressure reactor provided with stirrer, jacket heating and cooling, metering facilities for solid and liquid substances including alkylene oxides and also facilities for introduction of nitrogen and a vacuum system was dried by heating to 80° C. 145.4 kg of glycerol and 1000 g of a 50% strength aqueous imidazole solution were added and the reactor was made inert with nitrogen three times. The stirrer was started and the reactor was heated to 120° C. 354.0 kg of ethylene oxide were subsequently metered in (initial pressure: 2.5 bar). After a reaction time of 1.5 hours at 120° C., the remaining pressure was released and the mixture was stripped under reduced pressure for 30 minutes. This gave 485.3 kg of polyol having the following specifications:

| hydroxyl number | 527 mg KOH/g |
|---|---|
| viscosity | 275 mPas at 25° C. |
| water content | 0.006% |

### Comparative Example 1

572.4 g of terephthalic acid, 316.3 g of oleic acid, 447.9 g of diethylene glycol and 907.8 g of a worked up polyether alcohol A based on glycerol and ethylene oxide having an OH functionality of 3 and a hydroxyl number of 540 mg KOH/g and a residual alkalinity of <500 mg KOH/g and prepared using KOH catalysis were used, with the general method for preparing the polyester polyol being employed. This gave a polyesterol having an OH functionality of 2.49 and a hydroxyl number of 241 mg KOH/g.

### Comparative Example 2

601.9 g of terephthalic acid, 352.9 g of soybean oil, 461.4 g of diethylene glycol and 814.3 g of a worked up polyether alcohol A based on glycerol and ethylene oxide having an OH functionality of 3 and a hydroxyl number of 540 mg KOH/g and prepared using KOH catalysis were used, with the general method for preparing the polyester polyol being employed. This gave a polyesterol having an OH functionality of 2.49 and a hydroxyl number of 249 mg KOH/g.

### Comparative Example 3

668.2 g of terephthalic acid, 356.2 g of soybean oil, 501.6 g of diethylene glycol and 719.0 g of a worked up polyether alcohol B based on trimethylolpropane and ethylene oxide having an OH functionality of 3 and a hydroxyl number of 610 mg KOH/g and prepared using KOH catalysis were used, with the general method for preparing the polyester polyol being employed. This gave a polyesterol having an OH functionality of 2.49 and a hydroxyl number of 246 mg KOH/g.

### Comparative Example 4

214.3 g of terephthalic acid, 54.6 g of oleic acid, 218.9 g of diethylene glycol and 162.1 g of an unneutralized polyether alcohol A based on glycerol and ethylene oxide having an OH functionality of 3 and a hydroxyl number of 540 mg KOH/g and prepared using KOH catalysis were used, with the general method for preparing the polyester polyol being employed. This gave a 2-phase product which after neutralization of the KOH does not give a homogeneous product.

### Example 1 According to the Invention

572.4 g of terephthalic acid, 316.3 g of oleic acid, 447.9 g of diethylene glycol and 907.8 g of a polyether alcohol C based on glycerol and ethylene oxide having an OH functionality of 3 and a hydroxyl number of 540 mg KOH/g and prepared by the general method for preparing the polyetherol were used, with the general method for preparing the polyester polyol being employed. This gave a polyesterol having an OH functionality of 2.49 and a hydroxyl number of 252 mg KOH/g.

### Example 2 According to the Invention

601.9 g of terephthalic acid, 352.9 g of soybean oil, 461.4 g of diethylene glycol and 814.3 g of a polyether alcohol C based on glycerol and ethylene oxide having an OH functionality of 3 and a hydroxyl number of 540 mg KOH/g and prepared by the general method for preparing the polyetherol were used, with the general method for preparing the polyester polyol being employed. This gave a polyesterol having an OH functionality of 2.49 and a hydroxyl number of 248 mg KOH/g.

TABLE 1

| | | | | Catalysis | |
|---|---|---|---|---|---|
| | | Hydroxyl | | in the | |
| | | number | | preparation | Viscosity |
| | OH | (mg | PEOL | of the | 25° C. |
| | functionality | KOH/g) | component | PEOL | (mPa · s) |
| Comparative | 2.49 | 241 | A; glycerol and | neutralized | 2500 |
| example 1 | | | ethylene oxide | KOH | |
| Comparative | 2.49 | 249 | A; glycerol and | neutralized | 2640 |
| example 2 | | | ethylene oxide | KOH | |
| Comparative | 2.49 | 246 | B; | neutralized | 5086 |
| example 3 | | | trimethylolpropane | KOH | |
| | | | and ethylene | | |
| | | | oxide | | |
| Example 1 | 2.49 | 252 | C; glycerol and | imidazole | 2200 |
| according to | | | ethylene oxide | without | |
| the invention | | | | work-up | |
| Example 2 | 2.49 | 248 | C; glycerol and | imidazole | 2500 |
| according to | | | ethylene oxide | without | |
| the invention | | | | work-up | |

Table 1 shows that the polyesterols prepared by the process of the invention display no significant difference from the standard products (comparative examples).

Measurement Methods

A qualitative assessment of the nature of the surface of the rigid PUR foams was undertaken by removing the covering layer from a 1 m×2 m foam specimen and optically assessing the surfaces.

Determination of the Processability:

The processability was determined by examining foam formation during processing. Large blowing agent bubbles which burst at the foam surface and thus tear open the surface were referred to as “blow-outs” and the system could not be processed in a problem-free manner. If this unsatisfactory behavior was not observed, processing was problem-free.

Thickness

To determine the element thickness after foaming, a sandwich element having a 0.05 mm thick aluminum foil as covering layer material was produced by the double belt process and the element thickness is determined in the middle of the element 5 minutes after production.

Production of rigid polyurethane foams (Variant 1):

The isocyanates and the components which are reactive toward isocyanate were foamed together with the blowing agents, catalysts and all further additives at a constant mixing ratio of polyol to isocyanate of 100:190.

Polyol Component:
47.5 parts by weight of polyesterol as per examples or comparative examples
15 parts by weight of polyetherol having an OH number of ˜490 mg KOH/g and prepared by polyaddition of propylene oxide onto a sucrose/glycerol mixture as starter molecule
10 parts by weight of polyetherol comprising the ether of ethylene glycol and ethylene oxide and having a hydroxyl functionality of 2 and a hydroxyl number of 200 mg KOH/g
25 parts by weight of flame retardant trichloroisopropyl phosphate (TCPP)
2.5 parts by weight of stabilizer Niax Silicone L 6635 (silicone-containing stabilizer)
6.5 parts by weight of pentane S 80:20
about 2.3 parts by weight of water
1.5 parts by weight of potassium acetate solution (47% strength by weight in ethylene glycol)
about 1.1 parts by weight of dimethylcyclohexylamine

Isocyanate Component:

190 parts by weight of Lupranat® M50 (polymeric methylenedi(phenyl diisocyanate) (PMDI) having a viscosity of about 500 mPa*s at 25° C.)

50 mm thick sandwich elements were produced by the double belt process. The foam density was set to 38+/−1 g/l at a constant pentane content of 6.5 parts by varying the water content. The fiber time was also set to 25+/−1 s by varying the proportion of dimethylcyclohexylamine.

The results are summarized in Table 2 and Table 3

TABLE 2
Results of the experiments on production of 50 mm thick
sandwich elements by the double belt process

| | | Example 1 |
|---|---|---|
| | Comparative | according to |
| Polyester polyol: | Example 1 | the invention |
| Visual assessment | good | good |
| Processing | problem-free | problem-free |

TABLE 3
Results of the experiments on production of 50 mm thick
sandwich elements by the double belt process

| | | Example 2 |
|---|---|---|
| | Comparative | according to |
| Polyester polyol: | Example 2 | the invention |
| Visual assessment | good | good |
| Processing | problem-free | problem-free |

Tables 2 and 3 show that the rigid polyurethane foams produced by the process of the invention retain the good process properties.

Furthermore, 170 mm thick sandwich elements were produced by the double belt process using the systems. The foam density was set to 38+/−1 g/l at a constant pentane content of 6.5 parts by varying the water content. The fiber time was also set to 40+/−1 s by varying the proportion of dimethylcyclohexylamine.

The results are summarized in Tables 4 and 5:

TABLE 4
Results of the experiments on production of 170 mm thick
sandwich elements by the double belt process

| | | Example 1 |
|---|---|---|
| | Comparative | according to the |
| Polyester polyol: | example 1 | invention |
| Element thickness | 180 mm | 180 mm |
| after foaming | | |

TABLE 5
Results of the experiments on production of 170 mm thick
sandwich elements by the double belt process

| | | Example 2 |
|---|---|---|
| | Comparative | according to |
| Polyester polyol: | Example 2 | the invention |
| Element thickness | 182 mm | 181 mm |
| after foaming | | |

Tables 4 and 5 show that the dimensional stability of the polyurethane system is retained by the use of the polyesterol according to the invention.

Production of rigid polyurethane foams (Variant 2):

Furthermore, test plates were produced by the double belt process according to the following process for the production of a rigid polyurethane foam (Variant 2).

The isocyanates and the components which are reactive toward isocyanate were foamed together with the blowing agents, catalysts and all further additives at a constant mixing ratio of polyol component to isocyanate component of 100:170.

Polyol Component:
58 parts by weight of polyesterol as per Examples or Comparative Examples
10 parts by weight of polyetherol comprising the ether of ethylene glycol and ethylene oxide having a hydroxyl functionality of 2 and a hydroxyl number of 200 mg KOH/g
30 parts by weight of flame retardant trischloroisopropyl phosphate (TCPP)
2 parts by weight of stabilizer Tegostab B 8443 (silicone-comprising stabilizer)
10 parts by weight of n-pentane
1.6 parts by weight of formic acid solution (85%)
2.0 parts by weight of potassium formate solution (36% strength by weight in ethylene glycol)
0.6 part by weight of bis(2-dimethylaminoethyl)ether solution (70% by weight in dipropylene glycol)

Isocyanate Component:

170 parts by weight of Lupranat® M50

50 mm thick sandwich elements were produced by the double belt process. The foam density was set to 41+/−1 g/l at a constant formic acid content by varying the pentane content. The fiber time was also set to 25+/−1 s by varying the proportion of bis(2-dimethylaminoethyl)ether (70% by weight in dipropylene glycol).

The components were, as indicated, foamed together. The results of the surface assessment and the processability are summarized in Tables 6 and 7.

TABLE 6
Results of the experiments on production of 50 mm thick
sandwich elements by the double belt process

| | | Example 1 |
|---|---|---|
| | Comparative | according to |
| Polyester polyol: | Example 1 | the invention |
| Visual assessment | good | good |
| Processing | problem-free | problem-free |

TABLE 7
Results of the experiments on production of 50 mm thick
sandwich elements by the double belt process

| | | Example 2 |
|---|---|---|
| | Comparative | according to |
| Polyester polyol: | Example 2 | the invention |
| Visual assessment | good | good |
| Processing | problem-free | problem-free |

Tables 6 and 7 show that the rigid polyisocyanurate foams produced by the process of the invention retain the good processing properties.

Furthermore, 170 mm thick sandwich elements were produced by the double belt process using the systems. The foam density was set to 41+/−1 g/l at a constant formic acid content by varying the pentane content. The fiber time was also set to 40+/−1 s by varying the proportion of bis(2-dimethylaminoethyl)ether (70% by weight in dipropylene glycol).

The results are summarized in Tables 8 and 9:

TABLE 8
Results of the experiments on production of 170 mm thick
sandwich elements by the double belt process

| | | Example 1 |
|---|---|---|
| | Comparative | according to |
| Polyester polyol: | example 1 | the invention |
| Element thickness | 180 mm | 180 mm |
| after foaming | | |

TABLE 9
Results of the experiments on production of 170 mm thick
sandwich elements by the double belt process

| | | Example 2 |
|---|---|---|
| | Comparative | according to |
| Polyester polyol: | Example 2 | the invention |
| Element thickness | 182 mm | 181 mm |
| after foaming | | |

Tables 8 and 9 show that the dimensional stability of the polyurethane system can be retained by the use of the polyesterol according to the invention.

1. A polyester polyol comprising the esterification product of
a) from 10 to 70 mol % of a dicarboxylic acid composition comprising
a1) an amount of from 50 to 100 mol %, based on the dicarboxylic acid composition a), of an aromatic dicarboxylic acid or a mixture of aromatic dicarboxylic acids,
a2) an amount of from 0 to 50 mol %, based on the dicarboxylic acid composition a), of one or more aliphatic dicarboxylic acids,
b) from 2 to 30 mol % of one or more fatty acids and/or fatty acid derivatives,
c) from 10 to 70 mol % of one or more aliphatic or cycloaliphatic diols having from 2 to 18 carbon atoms or alkoxylates thereof,
d) from 2 to 50 mol % of a polyether alcohol having a functionality of greater than or equal to 2 and prepared by alkoxylation of a polyol e) having a functionality of greater than or equal to 2 in the presence of an amine as catalyst,
e) from 0 to 45 mol % of a polyether alcohol having a functionality of greater than or equal to 2 and prepared by alkoxylation of a polyol h) having a functionality of greater than or equal to 2 in the presence of a catalyst other than an amine,
f) from 0 to 50 mol % of a polyol having a functionality of greater than or equal to 2 selected from the group consisting of glycerol, trimethylolpropane, pentaerythritol,
where the mol % of components a) to f) add up to 100% and at least 200 mmol of polyols d) having an OH functionality of greater than or equal to 2, are reacted per kg of polyester polyol.

2. The polyester polyol according to claim 1, wherein the aromatic dicarboxylic acid a1) is selected from the group consisting of terephthalic acid, dimethyl terephthalate (DMT), polyethylene terephthalate (PET), phthalic acid, phthalic anhydride (PAn) and isophthalic acid.

3. The polyester polyol according to claim 1 or 2, wherein the aliphatic dicarboxylic acid a2) is selected from the group consisting of aliphatic dicarboxylic acids having from 4 to 6 carbon atoms.

4. The polyester polyol according to any of claims 1 to 3, wherein the fatty acid and/or the fatty acid derivative b) are selected from the group consisting of oleic acid, soybean oil and rapeseed oil.

5. The polyester polyol according to any of claims 1 to 4, wherein the aliphatic or cycloaliphatic diol having from 2 to 18 carbon atoms or alkoxylate thereof c) is diethylene glycol.

6. The polyester polyol according to any of claims 1 to 5, wherein the polyol g) and the polyol h) are each selected independently from the group consisting of glycerol, trimethylolpropane, pentaerythritol, polyethylene glycol (PEG) and mixtures thereof.

7. The polyester polyol according to any of claims 1 to 6, wherein the polyol g) has a functionality of greater than 2.

8. The polyester polyol according to any of claims 1 to 7, wherein the amine catalyst in d) is selected from the group consisting of DMEOA (dimethylethanolamine), imidazole and imidazole derivatives and mixtures thereof.

9. The polyester polyol according to any of claims 1 to 8, wherein the catalyst other than an amine in e) is selected from the group consisting of KOH, double metal cyanide (DMC) catalysts, carbenes.

10. The polyester polyol according to any of claims 1 to 9 having an average functionality of greater than or equal to 2.

11. The use of a polyester polyol according to any of claims 1 to 10 for producing a polyurethane.

12. A process for producing rigid polyurethane foams by reacting
A. at least one organic and/or modified organic diisocyanate and/or polyisocyanate with
B. at least one polyester polyol according to any of claims 1 to 10,
C. optionally at least one further polyester polyol,
D. optionally at least one polyetherol and/or at least one further compound having at least two groups which are reactive toward isocyanates,
E. optionally at least one chain extender and/or crosslinker,
F. at least one blowing agent,
G. at least one catalyst and also
H. optionally at least one further auxiliary and/or additive and
I. optionally at least one flame retardant.

13. A process for preparing a polyester polyol, wherein
a polyether alcohol (d) having a functionality of greater than or equal to 2 is prepared by alkoxylation of a polyol (g) having a functionality of greater than or equal to 2 in the presence of an amine as catalyst,
and a polyether alcohol (e) having a functionality of greater than or equal to 2 is optionally prepared by alkoxylation of a polyol (h) having a functionality of greater than or equal to 2 in the presence of a catalyst other than an amine and
from 2 to 50 mol % of the polyether alcohol (d) and
from 0 to 45 mol % of the polyether alcohol (e) and
from 0 to 50 mol % of a polyol (f) having a functionality of greater than or equal to 2 and selected from the group consisting of glycerol, trimethyloipropane, pentaerythritol, are reacted with
from 10 to 70 mol % of a dicarboxylic acid composition comprising (a1) from 50 to 100 mol %, based on the dicarboxylic acid composition (a), of an aromatic dicarboxylic acid or a mixture of aromatic dicarboxylic acids, (a2) from 0 to 50 mol %, based on the dicarboxylic acid composition (a), of one or more aliphatic dicarboxylic acids and
from 2 to 30 mol % of one or more fatty acids andlor fatty acid derivatives and
from 10 to 70 mol % of one or more aliphatic or cycloaliphatic diols having from 2 to 18 carbon atoms or alkoxylates thereof,
where the mol % of components (a) to (f) add up to 100% respectively and at least 200 mmol of polyols (d) having an OH functionality of greater than or equal to 2 are reacted per kg of polyester polyol.

14. The process for preparing a polyester polyol according to claim 13, wherein the aromatic dicarboxylic acid (a1) is selected from the group consisting of terephthalic acid, dimethyl terephthalate (DMT), polyethylene terephthalate (PET), phthalic acid, phthalic anhydride (PSA) and isophthalic acid.

15. The process for preparing a polyester polyol according to claim 13 or 14, wherein the aliphatic dicarboxylic acid (a2) is selected from the group consisting of aliphatic dicarboxylic acids having from 4 to 6 carbon atoms.

16. The process for preparing a polyester polyol according to any of claims 13 to 15, wherein the fatty acid and/or the fatty acid derivative (b) is/are selected from the group consisting of oleic acid, soybean oil and rapeseed oil.

17. The process for preparing a polyester polyol according to any of claims 13 to 16, wherein the aliphatic or cycloaliphatic diol having from 2 to 18 carbon atoms or alkoxylate thereof (c) is diethylene glycol.

18. The process for preparing a polyester polyol according to any of claims 13 to 17, wherein the polyol (g) and the polyol (h) are each selected independently from the group consisting of glycerol, trimethylolpropane, pentaerythritol, polyethylene glycol (PEG) and mixtures thereof.

19. The process for preparing a polyester polyol according to any of claims 13 to 18, wherein the polyol g) has a functionality of greater than 2.

20. The process for preparing a polyester polyol according to any of claims 13 to 19, wherein the amine catalyst in (d) is selected from the group consisting of DMEOA (dimethylethanolamine), imidazole and imidazole derivatives and mixtures thereof.

21. The process for preparing a polyester polyol according to any of claims 13 to 20, wherein the catalyst other than an amine in (e) is selected from the group consisting of KOH, double metal cyanide (DMC) catalysts, carbenes.

22. The process for preparing a polyester polyol according to any of claims 13 to 21, wherein the polyester polyol has an average functionality of greater than or equal to 2.

23. A polyester polyol which can be prepared by the process of any of claims 13 to 22.

24. The use of a polyester polyol which can be prepared by the process of any of claims 13 to 22 for producing a polyurethane.
